(19) United States
(12) Patent Application Publication
(10) Pub. No.: US 2009/0023194 A1
(43) Pub. Date: Jan. 22, 2009

(54) DEVICE AND METHOD FOR THE CULTIVATION AND GENERATION OF BIOLOGICAL MATERIAL IN A NUTRIENT MIST

(75) Inventors: Timo Schmidt, Stuttgart (DE); Lothar Just, Hechingen (DE)

Correspondence Address: KNOBBE MARTENS OLSON & BEAR LLP, 2040 MAIN STREET, FOURTEENTH FLOOR, IRVINE, CA, 92614, US

(73) Assignee: Eberhard-Karls-Universitaet Tuebingen Universitaetsklinikum, Tuebingen (DE)

(21) Appl. No.: 12/138,258

(22) Filed: Jun. 12, 2008

Related U.S. Application Data
(63) Continuation of application No. PCT/EP2006/012002, filed on Dec. 13, 2006.

(30) Foreign Application Priority Data
Dec. 14, 2005 (DE) DE102005061371.3

(51) Int. Cl. C12N 13/00; C12M 3/00

(52) U.S. Cl. 435/173.1; 435/289.1

(57) ABSTRACT

The present invention relates to a device and a method for the cultivation and generation of biological material.

### CROSS-REFERENCE TO RELATED APPLICATIONS

This application is a continuation of copending international patent application PCT/EP2006/012002 filed on Dec. 13, 2006 and designating the U.S., which was not published under PCT Article 21(2) in English, and claims priority of German patent application DE 10 2005 061 371.3 filed on Dec. 14, 2005, which is incorporated herein by reference.

### BACKGROUND OF THE INVENTION

1. Field of the Invention

The present invention relates to a device for the cultivation and generation of biological material, and a corresponding method.

2. Description of the Related Art

Numerous methods and systems for cultivating biological cells, cell assemblages and tissues are known in the prior art. With these known methods and systems, the biological cells are normally completely covered with a layer of liquid culture medium and incubated under defined conditions, i.e. at a particular temperature and in a fixed atmosphere, for a desired time.

These known cultivation systems have, however, a large number of disadvantages. The known systems are usually aimed at the cultivation of two-dimensional biological cell assemblages, called monolayer cultures. Cultivation of three-dimensional tissue constructs, organs, embryos etc. can by contrast be achieved with the known systems only conditionally, especially if this is to take place on a large scale. Thus, for example, with the known systems the gas supply to the individual biological cells of the three-dimensional tissue construct is rather unsatisfactory or achieved only with very great technical and financial outlay. With some of these systems, the so-called roller cultures, shear forces additionally occur and induce negative side effects on the behavior of the cells. With the known culture systems there is further known to be an increased risk of bacterial contaminations as the size and complexity thereof increase.

Furthermore, the known systems have limitations in relation to the present-day requirements of modern cell culture technologies. Most of the prior art systems are essentially aimed at pure cultivation of biological cells and tissues, i.e. merely “keeping alive” in vitro. They are only conditionally suitable in the context of the neogenesis or generation of biological tissue constructs or even whole organs by means of “tissue engineering”. Tissue engineering means according to the 1988 definition of the National Science Foundation (NSF) in the USA the application of principles and methods of engineering and life sciences toward fundamental understanding of structure-function relation-ships in normal and pathological tissue, and development of biological substitutes to restore, maintain or improve tissue function. In this connection, one principle of tissue engineering is based on specific cells being propagated outside the organism in cell culture, i.e., in vitro, and being differentiated in a targeted manner in the tissue assemblage and thus biological tissue constructs with desired properties being generated. One aim of tissue engineering is to replace damaged tissue in an organism by tissue constructs generated in vitro, with or without auxiliary constructs such as matrices, and thus to eliminate the losses of function caused by the cell damage.

### SUMMARY OF THE INVENTION

With this background, it is an object of the present invention to provide a device and a method both for the cultivation and for the generation of biological material in which the aforementioned disadvantages of the prior art are avoided. It is intended in particular that this device and this method make it possible for three-dimensional biological tissue constructs to be cultivated optimally, and for structural biological systems such as tissues, organs etc. to be generated and regenerated for example in the context of tissue engineering.

The object underlying the invention is achieved by a device for the cultivation and generation of biological material which comprises the following, namely: a unit for generating ultrasonic waves, a chamber able to receive a biological material and a culture medium and comprising an element which transmits ultrasonic waves and which comprises a first surface and a second surface, where the element is disposed in such a way that the first surface can make contact with the culture medium and the second surface can make contact with a medium which transmits ultrasonic waves and is disposed outside the chamber, and where the unit for generating ultrasonic waves is disposed outside the chamber in such a way that from these ultrasonic waves can be introduced into the medium which transmits ultrasonic waves, and be transmitted by the element into the culture medium.

The object underlying the invention is further achieved by a method for the cultivation and generation of biological material which comprises the following steps: (a) provision of biological material and culture medium in a chamber which comprises an element which transmits ultrasonic waves and which comprises a first surface and a second surface, where the element is disposed in such a way that the first surface can make contact with the culture medium and the second surface can make contact with a medium which transmits ultrasonic waves and is disposed outside the chamber; b) introduction of ultra-sonic waves into the medium which transmits ultrasonic waves in such a way that they are transmitted by the element into the culture medium.

In this connection, “cultivation” of biological material means according to the invention that the latter, after detachment from its natural or original environment, is maintained in vitro and—optionally—propagated and altered in its properties.

In contrast thereto, according to the invention “generation” means that, starting from biological starting material with particular starting properties, biological target material with desired target properties is formed newly and in a targeted manner. “Generation” thus means according to the invention the targeted generation of new biological structures or structural biological systems. It is possible to use for generating the target material the tissue engineering techniques with which the differentiation of the biological material is controlled, e.g. by influencing gene expression. It is possible by the generation according to the invention to form for example skin constructs from epithelial cells or three-dimensional cartilage replacement structures from cartilage cells, which can be used for pharmacological or else therapeutic purposes.

According to the invention, “biological material” refers to any undifferentiated stem and progenitor cells or differentiated cells of animal, human, plant, bacterial origin, assemblages of the aforementioned cells, organs, transplants, embryos, organisms etc. The biological material according to the invention may additionally be genetically manipulated by methods of molecular biology.

It is possible to provide as “unit for generating ultrasonic waves” an ultrasonic generator or ultrasound generator, respectively, which may comprise ceramic or semiconductor polymers as vibrating plates. The vibrating plates of the ultrasonic genera-tor can be made to vibrate with frequencies in the ultrasonic range and can be brought into contact with the medium which transmits ultrasonic waves. These vibrations may cause a wave motion in the medium which transmits ultrasonic waves. The ultrasonic generator is disposed in such a way that it can be introduced directly into the medium which transmits ultrasonic waves, but it is sufficient for the element which generates ultrasonic waves to be in contact with the medium which transmits ultrasonic waves. Ultrasonic generators are generally known in the prior art. Reference is made by way of example to EP 0 234 868 B1, JP 59 045 879 A and U.S. Pat. No. 4,332,105.

A “chamber” refers to any container which is suitable for receiving, cultivating, generating the biological tissue and for receiving a culture medium, and comprises sufficient space and—preferably—can be sealed toward the environment. The chamber must moreover be able to hold an aerosol mist of nebulized culture medium. In this connection, the skilled person is free to provide, besides a so-called “mist chamber” which can hold the culture medium in liquid form and serves to generate the aerosol mist of nebulized culture medium, one or more further chambers, for example “culture chambers” which are able to hold the biological material and are connected to the “mist chamber”. The chamber may furthermore be divided into individual subsidiary spaces, of which one fulfils the function of a “mist chamber” and another fulfils the function of a “culture chamber”. Chambers of these types are conventional in cell culture laboratories and are known to the skilled person, for example a cell biologist. A chamber is for example a space defined by walls, a roof and a base.

A “culture medium” refers to any medium known in the area of cell cultivation, such as, for example, DMEM Low Glucose, DMEM High Glucose, DMEM/Ham's F 12, Ham's F 10, Ham's F 12, Iscove's modified DMEM, L 15 Medium (Leibovitz's), Medium 199 with Earle's salts, Medium 199 with Hank's salts, Minimal Essential Medium Alpha Modification, PMI 1640, DMEM READY MIX, RPMI 1640 READY MIX, Thermostable Media; all obtainable from PAA Laboratories GmbH, Pasching, Austria.

An “element which transmits ultrasonic waves” means an element which can pass ultrasonic waves onward to the surrounding medium. For this purpose, the element which transmits ultrasonic waves has a material density which confers thereon preferably similar or most preferably the same acoustic properties as those of the surrounding medium. The natural frequencies of the element and of the surrounding medium are preferably similar and most preferably identical. If the surrounding medium has a high density, preferably an element with a likewise high density is employed; if the surrounding medium has a low density, preferably an element with a low density is employed. The element which transmits ultrasonic waves comprises according to the invention at least two sides or surfaces which are arranged such that the first surface can make contact with the culture medium and the second surface can make contact with the medium which transmits ultrasonic waves.

A “medium which transmits ultrasonic waves” is a medium in which waves can be generated by the impact of ultrasonic and can be passed onward to the surroundings. It is preferred in this connection for the medium to pass the ultrasonic waves onward to the surroundings with minimal loss. Examples of suitable media are liquids or gels which can be selected to have any desired properties in relation to the sound transmission for example via their specific density.

According to the invention, the unit for generating ultrasonic waves, for example the ultrasonic generator, is disposed outside the chamber and thus outside the culture medium. This has the advantage that contamination of the culture medium of the biological material by the ultrasonic generator is avoided, and operations in the chamber can take place where appropriate under sterile conditions. Furthermore, a harmful heat transfer by the ultrasonic generator to the culture medium and thus the biological material is substantially avoided. The temperature necessary for the particular cultivation or generation of the biological material can be controlled particularly well thereby.

The passing of the ultrasonic waves onward to the interior of the chamber and the culture medium therein takes place by means of the medium which transmits ultrasonic waves and of the element which transmits ultrasonic waves. The ultrasonic waves which are introduced into the medium which transmits ultrasonic waves impinge on the second surface, which is in contact with the latter, of the element which transmits ultrasonic waves. The element which transmits ultrasonic waves is activated thereby and passes the sound waves onward to the culture medium via the first surface which is in contact with the latter.

The passing of the ultrasonic waves onward into the culture medium produces “droplets” of the culture medium in the chamber in the form of a vapor, aerosol, mist or a nebulization. The droplets have in this case for instance a diameter which is about 0.01 to 10 μm, but may also be larger or smaller. The droplets generated by such a mist-forming method are preferably uniformly distributed in the chamber.

The object underlying the invention is thus completely achieved. The inventors provide for the first time a method for generating biological material which avoids the prior art problems mentioned at the outset and thus open up entirely new possibilities for cell culture technology.

Thus, especially in relation to the three-dimensional biological materials which are to be generated by means of tissue engineering, or else such two-dimensional biological materials where at least one radius of curvature is not infinite, the provision of the culture medium in the form of a medium vapor consisting of droplets is superior in several points to a conventional culture system in which the biological material is covered with a layer of medium. Exposure of the material to the medium vapor results in better gas diffusion in the tissue and thus for example an improved oxygen supply. Three-dimensional tissue constructs usually have a smaller surface area in relation to the stressed volume, leading to an unfavorable oxygen availability on normal cultivation by covering with a layer of medium. A further advantage is that, through the provision of droplets, the method according to the invention consumes less nutrient medium than conventional cultivation methods. Furthermore, contamination of the culture medium with bacteria is minimized by the exposure to medium vapor because they are damaged or even killed during the nebulization. A further advantage is that no shear forces occur as are known for example with roller cultures. There is no limit on the size of the tissue construct to be generated, as occur with conventional cultivation techniques through diverse demands on the system. The inventors have further realized that transplants cultivated or generated in the context of the method according to the invention retain their functionality even over a long culture time.

According to the invention, the unit for generating ultrasonic waves in the device is preferably designed so that it can form an ultrasonic focus. It is preferred in the method according to the invention for the ultrasonic waves to be introduced into the transmitting medium in step (b) in such a way that an ultrasonic focus is produced in the culture medium or in the medium which transmits ultrasonic waves.

These measures have the advantage that the extent of droplet formation in the culture medium can be controlled more easily. The ultrasonic waves are focused in the ultrasonic focus which represents the point of highest energy.

The unit for generating ultrasonic waves in the device according to the invention is preferably designed so that it is able to position the ultrasonic focus in the culture medium, more preferably can alter its position in the culture medium, and most preferably can alter its position in the culture medium in such a way that the focus coincides with the surface of the culture medium. In a preferred development of the method according to the invention it is preferred for the ultrasonic waves to be introduced into the culture medium in step (b) in such a way that the ultrasonic focus coincides with the surface of the culture medium.

An optimal nebulization of the culture medium depends decisively on the position of the ultrasonic focus in the culture medium. The possibility of flexible positioning and changing of position of the ultrasonic focus in the culture medium allows the intensity and the extent of the mist formation in the chamber to be regulated as desired. The displacement, which can preferably take place in the vertical but also in the horizontal direction, allows temperature peaks to be avoided in the medium, which is heated most in the area of the point of highest energy in the culture medium. Locating the ultrasonic focus directly on the surface of the culture medium achieves maximal nebulization.

The element which transmits ultrasonic waves is preferably disposed in the base of the chamber.

This measure creates the structural conditions for optimal arrangement of the element which transmits ultrasonic waves. In this connection, the element is inserted for example into a window which is provided in the base of the chamber, for example by pressing, screwing, welding, bonding or clamping. It is further possible for the element also to form the entire base or chamber. It is ensured in this case that the interior of the chamber is separated from the exterior of the chamber in a liquid and preferably air-tight manner, so that no medium which transmits ultrasonic waves can enter the chamber or culture medium escape from the chamber.

It is preferred according to the invention for the element which transmits ultrasonic waves to be a plate, diaphragm or a sheet and to consist of a material which transmits ultrasonic waves optimally. Examples of suitable materials are: ceramics, glass, minerals, metals, metal alloys and plastics, the plate or diaphragm preferably having a thickness which is between 0.005 mm and 1 cm, and most preferably 0.025 mm.

The latter embodiments and materials are particularly suitable, owing to their acoustic properties, for passing ultrasonic waves generated outside the chamber onward into the culture medium.

The transmitting properties of the element can be improved by the plate or the diaphragm being convex in the direction of the medium which transmits ultrasonic waves.

The transmitting properties of the element are particularly good when it has essentially the same density and the same ultrasonic transmission properties as the culture medium and/or the medium which transmits ultrasonic waves.

Under this condition, the ultrasonic waves are passed from the medium disposed outside the chamber onward into the culture medium inside the chamber with minimal loss, and optimal nebulization of the culture medium can be achieved.

In a preferred embodiment, the device further comprises a second chamber which is able to receive the medium which transmits ultrasonic waves, this chamber further preferably comprising a boundary wall which in turn comprises the element which transmits ultrasonic waves.

The design conditions for a device according to the invention are achieved advantageously with this measure. The second chamber, which can also be referred to as transmission chamber, can be disposed directly on the base of the cultivation chamber, so that the base of the latter simultaneously represents the roof of the second chamber. The base of the cultivation chamber thus provides the boundary wall of the second chamber. A unitary device is thus provided in a cost-effective manner.

The second chamber is preferably designed so that it is capable of at least partial reception of the unit for generating ultrasonic waves.

This embodiment makes it possible for the ultrasonic generator either to be introduced directly, or else its element which generates ultrasonic waves to be introduced into the medium which transmits ultrasonic waves. Partial reception can be achieved through the second chamber having an aperture which is suitable for inserting a for example rod-like element on whose end the element which generates ultrasonic waves is located.

The device according to the invention further comprises in a preferred embodiment a unit for generating physiological or/and physical stimuli acting on the biological material. It is preferred in the method according to the invention for a physiological or/and physical stimulation of the biological material to take place in a further step (c).

According to the invention, “physiological stimulation” refers to the induction of differentiation, proliferation or molecular development processes in the biological material by direct or indirect interaction with biological, chemical and physical factors. Examples of such factors are growth factors and culture medium factors, biological and nonbiological matrices, the ionic composition or the pH of the culture medium.

According to the invention, “physical stimulation” refers to the induction of differentiation, proliferation or molecular development processes in the biological material by direct or indirect action of physical forces and/or energies on the material. Examples of such forces and energies are: compressive, tensile, shear, centrifugal, centripedal, gravitational, microgravitational forces, kinetic energy, electrical pulses and waves, magnetism, temperature, etc.

Units for physiological and physical stimulation include mechanical, pneumatic, hydraulic systems, rotary drives, generators, magnets, heating devices, cultivation devices, natural biomaterials, artificial biomaterials etc. and are extensively available to the skilled person. The individual parameters of the stimulation, such as the nature of the stimulus, the duration and the pattern of stimulation are left to the skilled person and depend for example on the nature of the biological starting and target materials, the later purpose of use of the generated biological material, any experiments to be carried out on the biological material during the generation, etc.

The inventors combine for the first time in an inventive way knowledge from cell cultivation technology with that from modern cell biology and biotechnology. Thus, the methods disclosed in EP 0 234 868 B1, JP 59 045 879 A and U.S. Pat. No. 4,332,105 are methods for the pure cultivation of plant cells which provide no stimulation unit or comparable measures for genetic manipulation of biological material, and are therefore unsuitable for the generation according to the invention of biological material, especially of animal or human origin. It was particularly surprising that measures from known methods, such as the generation and provision of droplets of culture medium, can also be applied success-fully to the generation of biological structures by means of tissue engineering. Thus, the inventors have been able successfully to “cultivate” chicken embryos up to a specific stage of development, and all the measured physiological parameters confirmed normal development during the analyzed period. This was not to be expected from the data in the aforementioned documents, which are concerned with the mere cultivation of plant cells, owing to the biological complexity of embryos.

It is preferred with the device according to the invention for the unit for generating physiological stimuli to be achieved by a device for delivering growth factors into the culture medium. It is preferred in the method according to the invention for the physiological stimulation to be achieved by contacting the biological material with growth factors which are preferably provided in the culture medium.

The growth factors are advantageously delivered by this measure as physiological stimuli to the biological material via the culture medium. The delivery device can include for example a receptacle for the culture medium, which is preferably disposed outside the chamber and thus is easily accessible for adding the growth factors. A tube is preferably connected to the receptacle and can be passed through a peristaltic pump, and introduces the medium with the growth factors dissolved therein into the chamber. Since some growth factors have a limited lifespan, they can be added to the culture medium as often as desired and added in varying concentration. It will be appreciated that further delivery devices routinely used in biological or chemical laboratories can be employed.

As alternative or in addition, the unit for generating physiological stimuli in the device according to the invention can be achieved by a biological matrix in contact with the biological material, or/and by a non-biological matrix. In a preferred development of the method according to the invention, the physiological stimulation is achieved by contacting the biological material with a biological matrix or/and a non-biological matrix.

The growth factors can be introduced onto or into biological or non-biological matrices with which the biological material is incubated. It is ensured in this way that the growth factors reach the surface of the biological material and can induce intracellular differentiation and/or proliferation processes by interacting with specific surface molecules and receptors.

It is possible by this measure to control the generation process particularly advantageously through the choice of appropriate growth factors. According to the invention, “growth factors” refer in general to substances which control biological growth and development processes. Examples of such growth factors are: fibroblast growth factor (FGF), bone morphogenetic protein (BMP), keratinocyte growth factor (KGF), epidermal growth factor (EGF), nerve growth factor (NGF), insulin-like growth factor (IGF), trans-forming growth factor (TGF), tumor necrosis factor (TNF) etc. The choice and concentration of the particular growth factor, and the mode of stimulation depends on the desired biological material which is to be generated, and can be selected by the skilled person.

It is ensured according to the invention that the growth factors are effectively delivered to the biological material, and the physiological stimulation of the latter is thus achieved in a simple way.

The alternative physiological stimulation by contacting the biological material with a biological matrix or/and a non-biological matrix ensures that the matrices are in direct contact with the biological material and form a composite with the latter. Knowledge of modern cytobiology and developmental biology is advantageously utilized by this measure for stimulating the biological material. Thus, it is known to those skilled in the art that the expression and the attainment of specific tissue properties depend substantially on the anchoring of the cells and thus on the surface of the substrate during the cultivation. The stimulation via matrices thus has an influence on the differentiation and proliferation processes, the shape, the firmness and the elasticity of the biological material to be generated.

Suitable biological matrices are all biogenic substances available to the skilled person, such as, for example, collagens, fibrin, chitin, laminins, fibronectin, hyaluronic acid or hydroxyapatite. Targeted control of the material to be generated is possible via the choice of the biogenic substances. With the method according to the invention it is possible for example to generate a three-dimensional cartilage substitute via bovine collagen of type I as biogenic matrix substance by contacting with cartilage cells as biological starting material. A further suitable biological matrix is the so-called extracellular matrix (ECM) or a cell-free tissue framework from ECM, which is obtained from native tissues by methods known to the skilled person. In these methods, the complex three-dimensional disposition of the extracellular matrix proteins is retained and thus ensures that the differentiation of the biological tissue is as close to in vivo as possible. These matrices are already being produced from heart valves, the small intestine, the esophagus and the dermis; cf. Tian et al. (2005), Effect of small intestinal submucosa on islet recovery and function in vitro culture, Hepatobiliary Pancreat. Dis. Int. 4(4), pages 524-9; Ruszczak, Z. (2003), Effect of collagen matrices on dermal wound healing, Adv. Drug. Deliv. Rev. 55(12), pages 1595-611; Zhu et al (2005), The growth improvement of porcine esophageal smooth muscle cells on collagen-grafted poly(DL-Lactide-co-Glycolide) membrane, J. Biomed. Mater. Res. B. Appl. Biomater. 75(1), pages 193-9; Schenke-Layland et al. (2003), Complete dynamic repopulation of decellularized heart valves by application of defined physical signals—an in vitro study, Cardiovasc. Res. 60(3), pages 497-509. It is advantageous in this connection that both allogeneic and xenogeneic, i.e., animal-derived, materials can be used because they usually lead only to weak or no immune responses, because the cellular constituents are removed and the extracellular matrix proteins are transformed by the biological tissue which is in contact.

Suitable non-biological matrices are for example polyanhydrides, polyphosphazines, polyamino acids, polyactide derivatives and polyglycol derivatives, which may be biodegradable or non-biodegradable.

Suitable according to the invention as physical stimuli acting on the biological material are those selected from the group consisting of: tensile forces, compressive forces, shear forces, centrifugal forces, centripedal forces, gravitational forces, microgravitational forces. Targeted generation of biological material with desired properties such as, for example, bone and cartilage structures, myocardial tissue, smooth muscle tissue and skeletal muscle tissue is possible by means of such stimuli. It has thus been reported by those skilled in the art that biological cells or tissues or organs can be altered or differentiated in a targeted manner by mechanical stimulation; cf. M. Benjamin and B. Hillen (2003), Mechanical Influences on Cells, Tissues and Organs—, Mechanical Morphogene-sis', Eur. J. Morphol. 41, pages 3-7. The choice of the forces chosen in each case, and the strength, duration and pattern of the stimulus depends on the desired biological materials to be generated and can be selected by the skilled person.

As alternative or in addition it is possible to provide as physical stimuli electrical current, a magnetic field, weightlessness, oxygen partial pressure, sound waves, capillary forces and adhesion forces. It is also known that these stimuli are capable of targeted control of differentiation of biological tissue. Thus, for example, it is known that the contractile properties of muscle tissue can be altered in a controlled manner by stimulation with certain electrical pulses.

The device according to the invention can comprise as unit for generating physical stimuli preferably a pneumatic, hydraulic or mechanical drive which sets the biological material in motion.

A physical stimulus is advantageously transmitted to the biological tissue with this measure. The pneumatic or hydraulic drive can in this case be disposed inside or outside the chamber and be connected to a cell carrier on which the biological material is disposed. By setting the carrier in motion, the motion impulse is transmitted to the biological material. The form of motion and frequency can be selected by the skilled person and includes translational, rotational, oscillatory, shaking movements and the like.

With the device according to the invention it is preferred for the unit for generating physical stimuli to be achieved by the chamber being a cushion whose shape can be altered by applying pressure.

The application of pressure can in this case take place for example by introducing a gaseous, liquid or vapor medium, for example the culture medium itself, into the cushion. It is advantageously possible in such a cushion for both the exposure to vapor and the physical stimulation of the biological tissue to take place. The biological material which is preferably disposed on the inner wall of the cushion or on a further membrane located inside the cushion is appropriately physically stimulated by the change in shape of the cushion. The pressure inside the cushion can be varied as desired, so that both superatmospheric and subatmospheric pressure systems can be provided. It is also possible by targeted design of the cushion to control the form of the motion transmitted to the biological material. Furthermore, it is also possible to exert a force on the cushion by putting liquid, gelatinous or gaseous substances in an adjoining chamber and applying superatmospheric or subatmospheric pressure thereto so that they exert a stimulus on the cushion.

The element which transmits ultrasonic waves is inserted in the base of the cushion itself and is in contact with the medium which transmits ultrasonic waves. The lower part or the base of the cushion may furthermore consist entirely of the element which transmits ultrasonic waves.

In an alternative variant of the device according to the invention, the unit for generating physical stimuli is achieved by a rotary drive which sets the biological tissue in rotation.

The biological material is advantageously exposed by this measure to centrifugal or centripedal forces which are able to influence the pattern of expression of the biological cells and thus their differentiation and/or proliferation rate. The biological tissue can in this variant be disposed in the interior of the chamber in a centrifuge drum which can be set in rotation at any speed by a drive motor.

Alternatively, the unit for generating physical stimuli in the device according to the invention may comprise means which are in contact with the biological material and exert mechanical pressure thereon, or a gas or liquid nozzle via which gas or liquid pressure is exerted on the biological material.

It is possible with this variant for the biological material to be physically stimulated in a different way and thus for its development to be influenced in a different way.

Further alternatives consist of achieving the unit for generating physical stimuli by a voltage generator or a magnet which respectively delivers electrical pulses to the biological material or exposes the biological material to a magnetic field.

A further possibility is for the unit for generating physical stimuli in the device according to the invention to comprise contractile biological cells which are in contact with the biological material.

This variant has the advantage that no further mechanical design arrangements are necessary on the device; on the contrary, the stimulation unit is achieved by “biological units”. The contractile cells can by contrast be supplied with culture medium and cultivated in the same way as the biological material in the chamber.

It is preferred for the device according to the invention to comprise a temperature control to control the temperature of the culture medium.

It is possible with this measure advantageously to bring the culture medium to the desired temperature, irrespective of the room temperature, so optimal conditions for generating tissue constructs can be set up.

It is further preferred for the device according to the invention to comprise a gas delivery for delivering gas to the biological tissue.

This measure ensures that the biological material is adequately supplied with the gases necessary for generating tissue constructs, such as, for example, oxygen or carbon dioxide. It is possible in this case for the device according to the invention to comprise a measuring probe to determine physiological parameters which serve, for example, for permanent monitoring of the amounts of gas and the pressure of gas, the glucose concentrations, lactate concentrations etc. An optionally provided control unit serves to control the pressure of gas and the amount of gas in the chamber. Measuring probes and control units of these types are known to the skilled person.

The method according to the invention can advantageously be carried out with the device according to the invention.

A further aspect developed by the inventors relates to a device for generating biological material which comprises the following, namely: a chamber which can receive biological material, and a unit for introducing droplets of a culture medium into the chamber, where the device also comprises a unit for generating physiological or/and physical stimuli acting on the biological material.

The method according to the invention can particularly advantageously be carried out with this device. No such device has to date been available in the state of the art. Thus, for example, the devices from EP 0 234 868 B1, JP 59 045 879 A and U.S. Pat. No. 4,332,105 which were mentioned at the outset, do not provide a unit for generating physiological or/and physical stimuli acting on the biological material, and in particular do not make it possible to generate biological material according to the invention. The known devices are, on the contrary, set up for pure cultivation of plant cells or animal monolayer cultures.

In the further device, the unit for generating physiological stimuli is preferably achieved by a device for delivering growth factors into the culture medium or, alternatively, by a biological matrix which is in contact with the biological material or/and a non-biological matrix.

In the further device, the unit for generating physical stimuli can preferably comprise a pneumatic or hydraulic or mechanical drive which sets the biological material in motion.

In the further device, the unit for generating physical stimuli may alternatively or additionally include a cushion in which the biological material is generated and whose shape can be altered by applying pressure, the cushion preferably being configured so that the droplets of the culture medium can be introduced into the interior of the cushion.

In the further device, the unit for generating physical stimuli may further comprise a rotary drive which sets the biological material in rotation, or means in contact with the biological material and exerting mechanical pressure thereon.

The unit for generating physical stimuli of the further device may also comprise a gas or liquid nozzle through which gas or liquid pressure is exerted on the biological material.

In the further device, the unit for generating physical stimuli may alternatively or additionally comprise a voltage generator which delivers electrical pulses to the biological material, or comprise a magnet which exposes the biological material to a magnetic field.

In the further device, the unit for generating physical stimuli may other-wise or additionally comprise contractile biological cells in contact with the biological material.

In a development of the further device, the unit for generating droplets comprises an ultrasonic nebulizer.

This measure has proved to be particularly suitable for generating liquid droplets. It is possible to provide for this purpose an ultrasonic generator which is introduced directly into the culture medium in the interior of the chamber and causes the latter to vibrate, by actuating a piezoceramic element with frequencies in the ultrasonic range, so that the wave motions caused thereby in the medium leads to formation of a mist of the medium in the interior of the chamber. Alternatively, the ultrasonic generator may comprise a nozzle or a vibrating head. Culture medium can be delivered to this vibrating head so that the former is nebulized by the vibration of the vibrating head and forms a mist in the chamber. In a further variant, the ultrasonic nebulizer can nebulize the medium outside the chamber in a subsidiary chamber. The mist which is generated is in this variant sup-plied to the actual culture chamber only after the nebulization. Ultrasonic nebulizers are generally known in the state of the art. Reference is made by way of example to EP 0 234 868 B1, JP 59 045 879 A and U.S. Pat. No. 4,332,105 already mentioned.

A temperature controller to control the temperature of the culture medium, and a gas supply to supply gas to the biological material, can be provided on the further device.

The inventors have additionally developed a further method for the cultivation and generation of biological material, which comprises the following steps: (a) provision of biological material; (b) introduction of the biological material into a chamber; (c) introduction of droplets of a culture medium into the chamber in such a way that the biological material comes into contact with the droplets, and (d) physiological or/and physical stimulation of the biological material.

The physiological stimulation can be achieved in the further method by contacting the biological material with growth factors which are preferably provided in the culture medium.

The physiological stimulation can furthermore be achieved in the further method by contacting the biological material with a biological matrix or/and a non-biological matrix.

The physical stimuli which act on the biological material in the further method are preferably selected from the group consisting of: tensile forces, compressive forces, shear forces, centrifugal forces, centripedal forces, gravitational forces, microgravitational forces, electric current, magnetic field, weightlessness, oxygen partial pressure, soundwaves, capillary forces, adhesive forces.

Concerning the features and advantages of the further device developed by the inventors and of the further method, the statements made above in connection with the device according to the invention and the method according to the invention apply correspondingly.

It will be appreciated that the features mentioned above and to be explained below can be used not only in the particular combinations indicated but also in other combinations or alone without departing from the scope of the present invention.

The invention is now explained in more detail by means of exemplary embodiments which have a purely illustrative nature and do not restrict the scope of the invention. In this connection, reference is made to the appended figures.

### BRIEF DESCRIPTION OF THE DRAWINGS

FIG. 1 shows a diagrammatic overview (A) of the further device with a first embodiment of the ultrasonic generator (B);

FIG. 2 shows a diagrammatic overview (A) of the further device with a second embodiment of the ultrasonic generator (B);

FIG. 3 shows a diagrammatic overview of a variant of the further device in which the mist is produced in a separate chamber and is delivered to the culture chamber;

FIG. 4 shows diagrammatic overviews of the further device which is introduced into a cell culture incubator;

FIG. 5 shows different variants of cell carriers;

FIG. 6 shows examples of various biological materials;

FIG. 7 shows a diagrammatic overview of the further device with a matrix component and growth factors as physiological stimuli;

FIG. 8 shows a diagrammatic overview of the further device with a first embodiment of a unit for physical stimulation of the biological material;

FIG. 9 shows various further embodiments of the unit for physical stimulation of the biological material as component of the further device;

FIG. 10 shows various embodiments of the unit for physical stimulation of the biological material as component of the device according to the invention;

FIG. 11 shows a diagrammatic overview of embodiments of the device according to the invention as two-chamber system;

FIG. 12 shows a further diagrammatic overview of embodiments of the device according to the invention in which the chamber is realized by a cushion, and the position of the ultrasonic focus can be altered;

FIG. 13 shows a diagrammatic representation of an embodiment of a cushion for physical stimulation and exposure of the biological material to medium vapor;

FIGS. 14, 16 and 17 show experimental demonstrations of the physiological functioning and vitality of various biological materials by the method according to the invention, and

FIG. 15 shows control of bacterial contamination by the method according to the invention.

### DETAILED DESCRIPTION OF THE PREFERRED EMBODIMENT

### Example 1

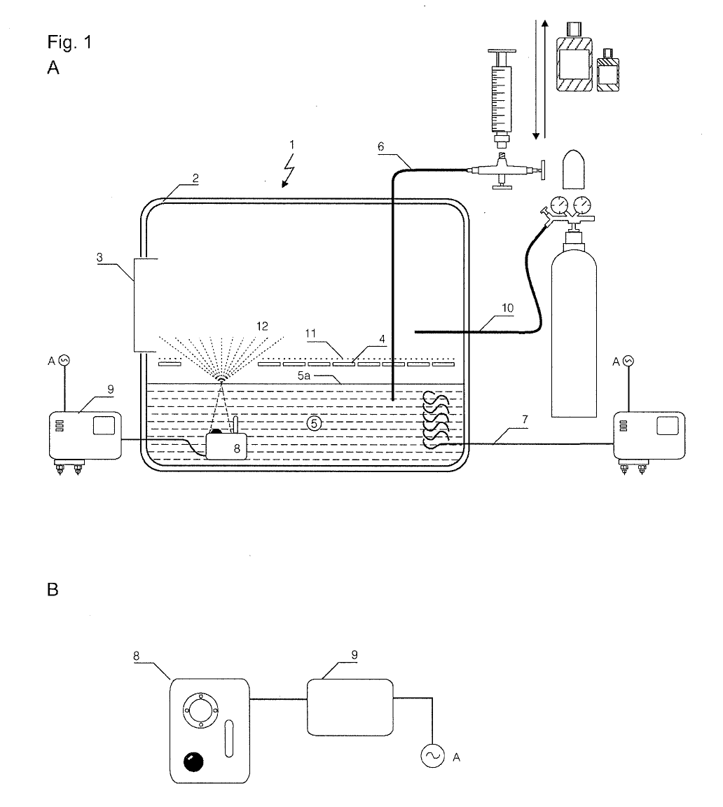

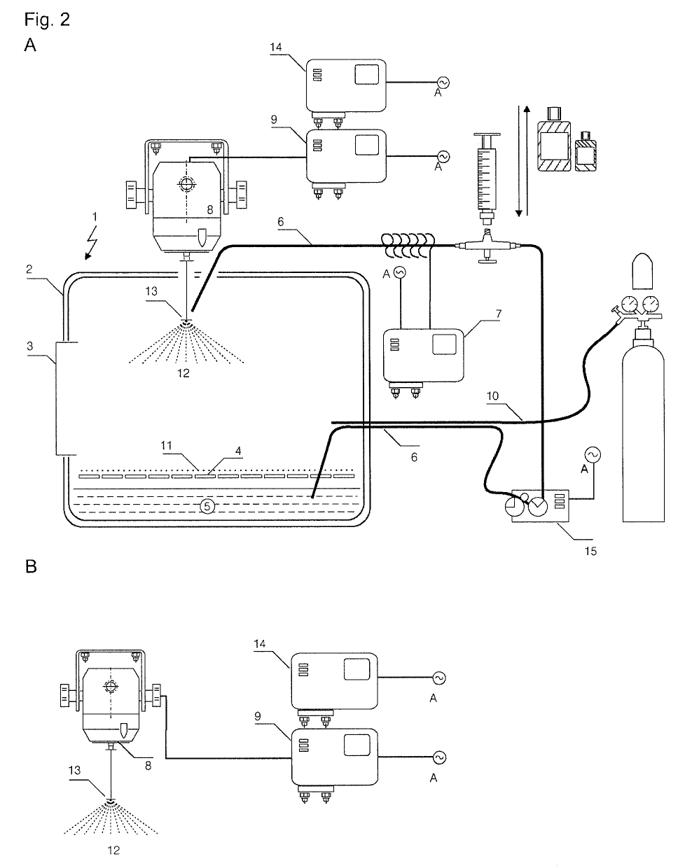

FIG. 1A depicts a first embodiment of a tissue generating device 1. This comprises a chamber 2 in the form of a customary culture vessel. An aperture flap 3 attached on the side allows access to the interior of the chamber 2. A carrier 4 is present in the interior of the chamber 2. Culture medium 5 is present over the base of the chamber 2 and can be introduced in a controllable manner into the interior of the chamber 2 via a medium delivery and removal 6 which is disposed outside the chamber 2 and which consists of a reservoir for the culture medium 5 and a tube. The temperature of the culture medium 5 can be adjusted by a heating coil of a temperature-control unit 7. The surface of the culture medium 5 forms a liquid edge 5a in the interior of the chamber 2.

An ultrasonic generator 8 is located on the base of the chamber 2 within the culture medium 5 and is connected to a voltage and frequency controller 9. The ultra-sonic generator 8 and the voltage and frequency controller 9 are depicted in detail in the partial picture of FIG. 1B. The ultrasonic generator (8) comprises a piezoceramic vibrator. The piezoceramic vibrations are brought about by a high-frequency alternating voltage. Ultrasonic waves are formed and have their maximum strength at a particular height of liquid and bring about the formation of an ultrasonic spout. Small liquid droplets—the aerosol or a mist 12—become detached from this ultrasonic spout and can then be used for the desired applications. The chamber 2 is therefore also referred to as mist chamber. A particular liquid level, for example of 55 mm with an accuracy of, for example, ±5 mm should preferably be kept constant. This is possible for example by a float switch or by the overflow principle. The amount of aerosol or mist 12 generated can be altered by an external control voltage or a potentiometer.

Any desired gases such as, for example, oxygen and carbon dioxide can be introduced into the chamber 2 through a gas delivery 10.

Biological material 11, for example biological cells, is present disposed on the carrier 4. When the ultrasonic generator 8 starts to operate there is formation, as explained above, of the mist 12, consisting of fine droplets of the culture medium 5, in the chamber 2. Growth factors can be present in the culture medium 5 as physiological stimuli and can be admixed in the desired concentration via the medium delivery and removal 6. The material 11 is optimally supplied with culture medium 5 and, where appropriate, the growth factors dissolved therein via the mist 12.

The duration of the dusting or stimulation of the material 11 can be varied as desired and depends in particular on the nature of the biological material 11 and of the tissue constructs to be generated.

FIG. 2A depicts, with use of the corresponding reference numbers, an alternative embodiment of the tissue generating device 1 in which the ultrasonic generator 8 is disposed outside the chamber 2, and a vibrating head 13 extends from the ultrasonic generator 8 through an aperture in the roof of the chamber 2 into the interior of the chamber to generate the mist 12 of culture medium 5. The medium delivery and removal 6 guides the culture medium 5 directly onto the vibrating head 13.

This variant of the ultrasonic generator is depicted in detail in the partial picture in FIG. 2B. A piezoceramic element converts electrical waves into mechanical vibrations. The liquid or the culture medium 5 which is applied without pressure to the vibrating body forms capillary waves from which a mist of very fine drops is discharged (mist 12).

Both the variants depicted in FIG. 1A and in FIG. 1B have various advantages: very low expenditure of energy through delivery of the medium 5 without pressure, low electrical power requirement, uniform nebulization of volumetric flows which may change from a maximum to approximately zero, virtually constant spectrum of drops over the entire range of volumetric flow, high operational reliability because virtually free of blockages, low characteristic velocity of the drops and thus the stream can be influenced satisfactorily, operationally reliable oscillator.

In the embodiment of the tissue generating device 1 depicted in FIG. 2, the medium delivery and removal 6 additionally comprises a peristaltic pump 15 by which the culture medium 5 can also be removed in a controlled manner from the chamber 2, so that a flow-through system is provided.

The heating coil of the temperature control unit 7 is in this variant placed outside the chamber 2 on the tube of the medium delivery and removal 6 which delivers the culture medium 5 to the chamber 2.

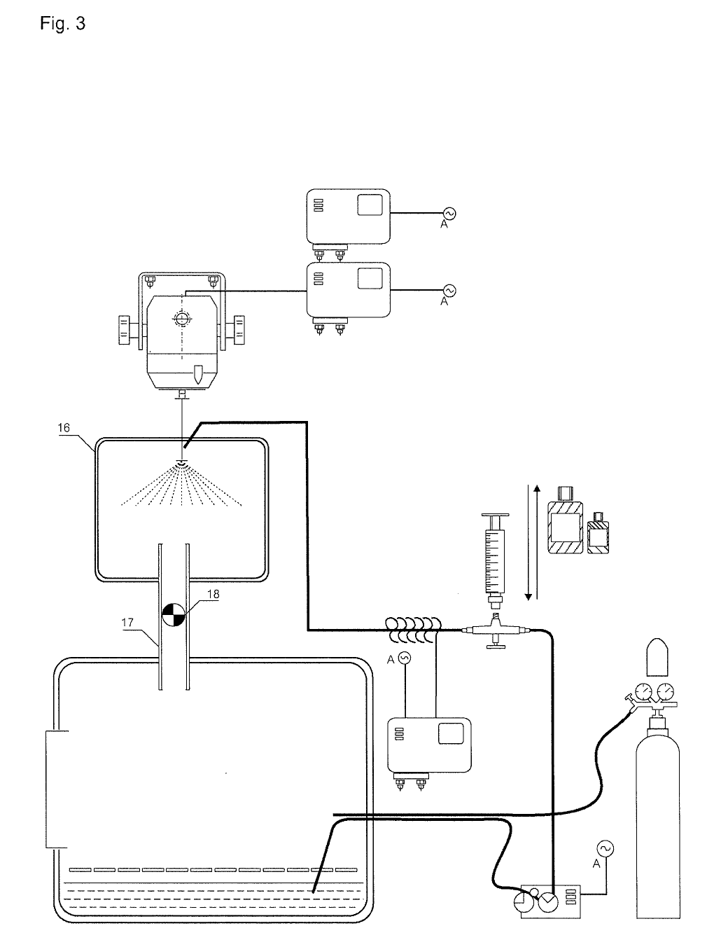

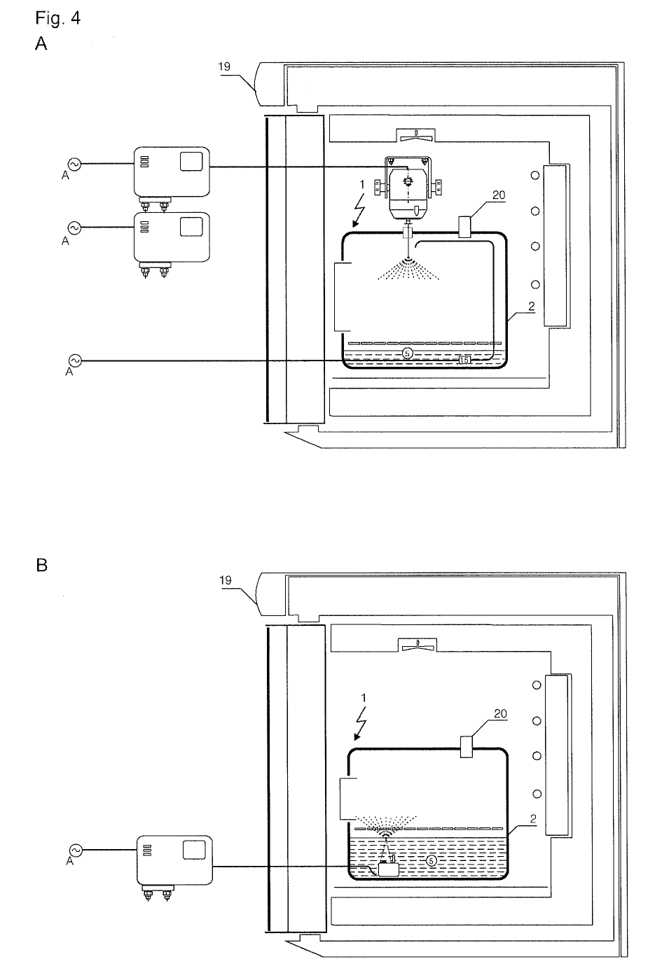

FIG. 3 depicts a further embodiment of the tissue generating device 1 in which the mist 12 is generated outside the chamber 2 in a separate chamber 16 and then introduced into the chamber 2 via a connecting unit 17 in which a fan 18 is disposed. This design alternative has the advantage that one ultrasonic generator 8 can be used for a plurality of chambers 2. Simplified maintenance of the ultrasonic generator 8 is also possible; the ultrasonic generator 8 can be placed outside the chamber 2 so that conventional incubators can be used as chamber 2, and the nebulization serves only as additional system which cooperates with present. The risk of contamination is further reduced with this variant.

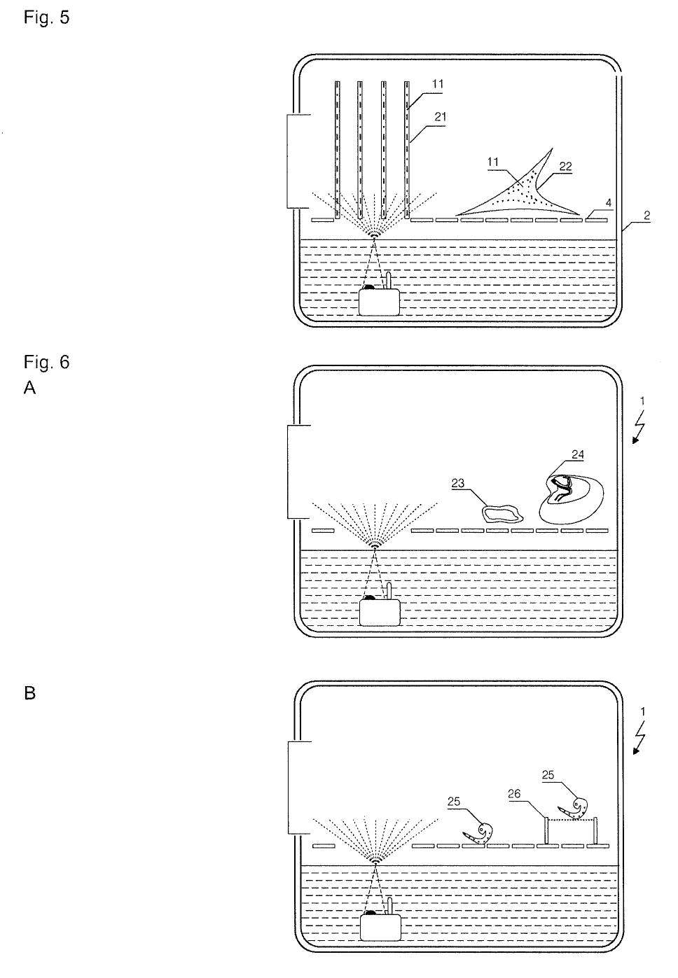

FIG. 4A and FIG. 4B depict further variants of the tissue generating de-vice 1 in which the latter is introduced into a cell culture incubator 19, by which the temperature and gas supply is controlled. A separate temperature control unit 7 and, where appropriate, gas delivery 10 can be dispensed with in this variant.

In this variant, a filter 20 is provided in the roof of the chamber 2 and has the function of making it possible for gas to diffuse to maintain the atmosphere generated in the cell culture incubator 19 as far as the actual chamber 2. Separate conditioning of the chamber 2 is thus unnecessary. The filter 20 also ensures sterile sealing of the chamber 2.

The partial picture in FIG. 4A shows the variant of the tissue generating device 1 in which a vibrating head 13 is provided as component of the ultrasonic generator 8, whereas the partial picture in FIG. 4B shows the ultrasonic generator 8 located in the culture medium 5. The partial picture of FIG. 4A further shows a variant of the medium delivery and removal 6 in which the peristaltic pump 15 is located inside the chamber 2 and the culture medium 5 is guided directly in a short circuit from the base of the chamber 2 to the vibrating head 13.

FIG. 5 depicts, besides the horizontally disposed carrier, variants of further carriers, namely a cell carrier 21 which is disposed vertically in the interior of the chamber 2, and a three-dimensionally curved cell carrier 22 on which the biological material 11 is disposed.

FIG. 6A depicts as biological material tissues or tissue constructs 23, and organs 24, which can be cultivated or generated with the tissue generating device 1. The partial picture in FIG. 6B shows embryos 25 which can be applied either directly to the carrier 4 or to a carrier membrane 26 and can be cultivated and generated with the tissue generating device 1.

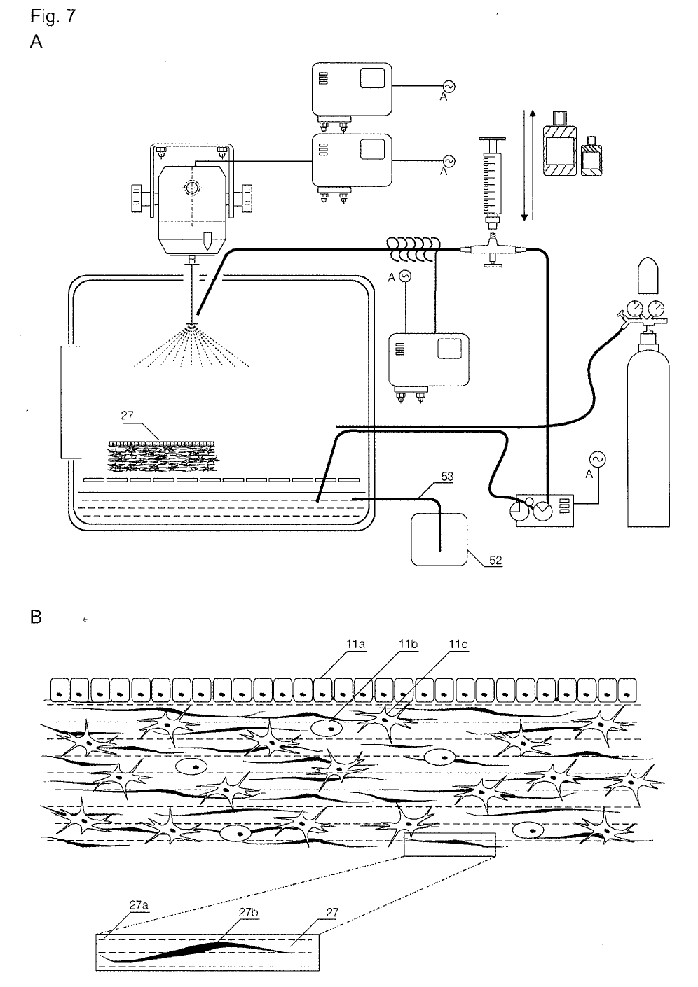

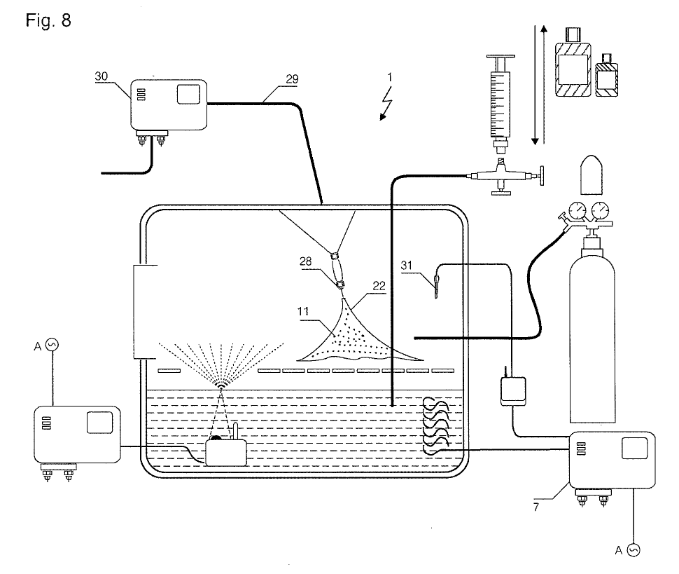

FIG. 7A shows the tissue generating device 1 which comprises a matrix component 27 which physiologically stimulates the biological material 11. As depicted in detail in the partial picture in FIG. 7B, the matrix component 27, which may be a biological or non-biological matrix 27a which may include cell proliferation and cell differentiation-promoting growth factors 27b, is in direct contact with the biological material 11 and thus mediates the physiological stimuli. The matrix component 27 may be in contact with different cell types 11a, 11b, 11c.

As alternative or in addition, the growth factors are introduced into the chamber 2 via a separate unit or device. Thus, the growth factors are provided in a container 52 in dissolved form and introduced into the interior of the chamber 2 or into the culture medium 5 via a delivery line 53. The medium is then passed via a circuit to the ultrasonic generator 8 which nebulizes the medium with the growth factors. The growth factors then come into contact with the biological material 11.

FIG. 8 shows a further variant of the tissue generating device 1 which comprises a pneumatic drive 28 which is connected via a compressed air line 29 to a compressed air generator 30. The pneumatic drive 28 is connected in the interior of the chamber 2 to the three-dimensionally curved cell carrier 22. When compressed air is applied to the pneumatic drive 28 via the compressed air generator 30 and the compressed air line 29, the pneumatic drive executes a translational movement which leads to movement of the three-dimensionally curved cell carrier 22 and thus to a physical stimulation of the biological material 11 disposed thereon.

In FIG. 8 there is also disposed a temperature sensor 31 which is connected to the temperature control unit 7 and provides information to the latter so that if the temperature falls below a set value the heating coil of the temperature control unit 7 brings the culture medium 5 back to the desired temperature.

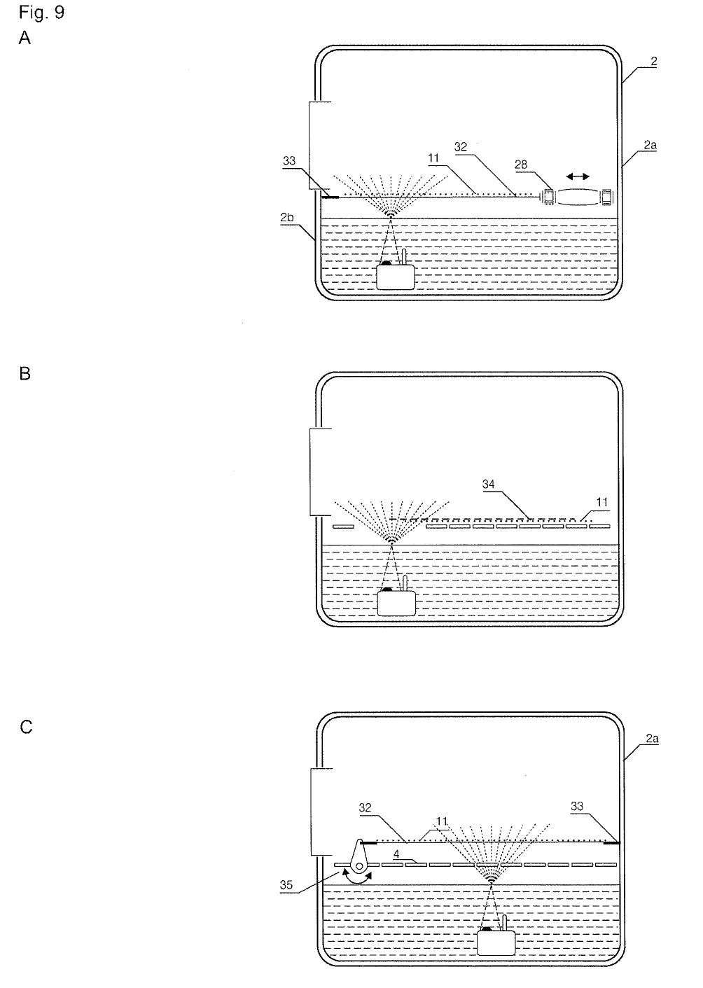

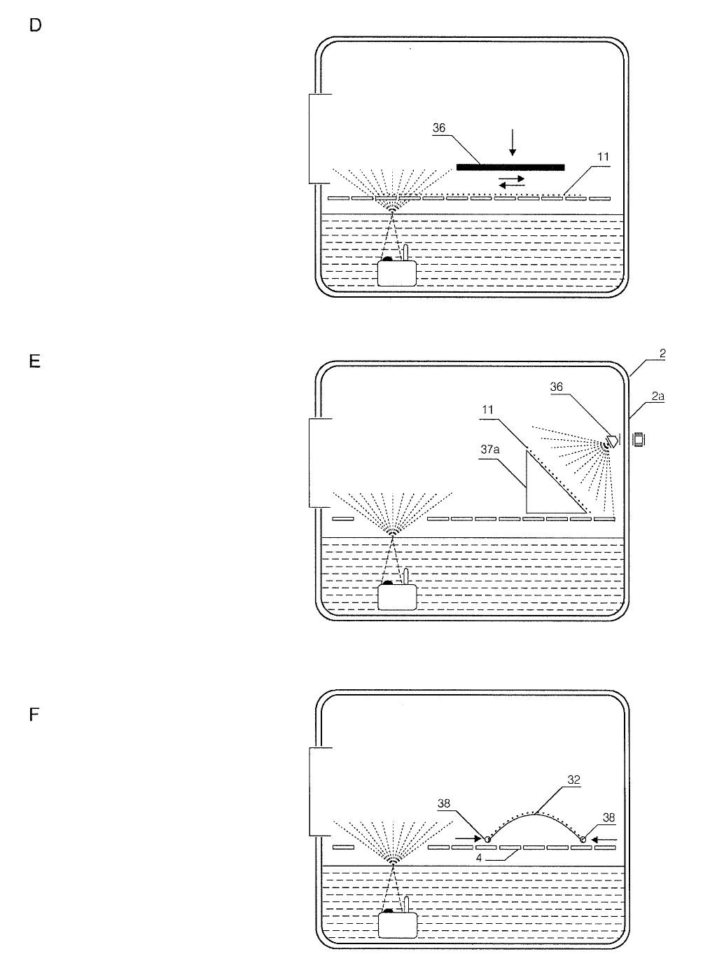

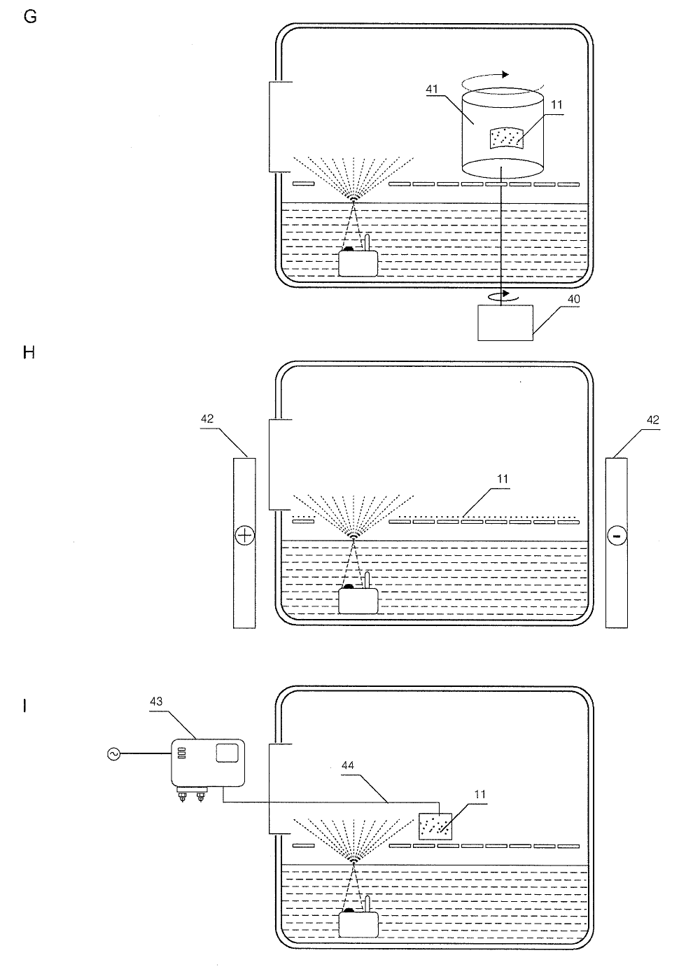

FIG. 9 depicts further variants of the unit for generating physical stimuli. The partial picture in FIG. 9A shows the pneumatic drive 28, which may also be a hydraulic drive, which is fixed on one side to the wall 2a of the chamber 2. The other side of the pneumatic drive 28 is connected to a flexible carrier 32 whose other end is connected to the wall 2b of the chamber 2 which is opposite to the wall 2a. The biological material 11 is disposed on the flexible carrier 32. When the pneumatic drive 28 starts to operate, it executes a translational movement which is indicated by the double arrow. This movement is transmitted to the flexible carrier 32 which is likewise set in motion, whereby the biological material 11 disposed thereon is physically stimulated.

As is evident from this partial picture in FIG. 9A, it is possible with the tissue generating device 1 for the biological material 11 to be simultaneously physically and physiologically stimulated, specifically by adding growth factors to the culture medium 5 for example in this embodiment.

The partial picture in FIG. 9B shows a variant of the physical stimulation of the biological material 11. In this variant, contractile cells 34 are provided in direct contact with the biological material 11 disposed on the carrier 4. The motion due to the contraction of the contractile cells 34 is transmitted as physical stimulus to the material 11.

It is possible in this variant for physiological stimuli to be provided at the same time by the contractile cells 34, specifically via the extracellular matrix of the contractile cells 34, which then corresponds to the matrix component 27.

The partial picture in FIG. 9C depicts a mechanical drive 35 as unit for generating physical stimuli. This is in articulated connection on its lower side to the carrier 4 and with the opposite side thereto to one end of the flexible carrier 32 on which the biological material 11 is disposed. The flexible carrier 32 is connected by its other end to the wall 2a of the chamber 2. When the mechanical drive 35 starts to operate, it executes a swinging movement, which is indicated by the double arrow. The flexible carrier 32 is thereby set in corresponding motion which is transmitted as physical stimulus to the biological material 11.

The partial picture in FIG. 9D depicts a further mechanical drive 36 which is in contact with the material 11 and exerts thereon mechanical pressure or mechanical shear stress, as indicated by the arrows.

The partial picture in FIG. 9E depicts as unit for generating physical stimuli a gas or liquid nozzle 37 which is fixed on the wall 2a of the chamber 2 and delivers liquid or gas to the biological material 11 with a desired pressure and thus causes pressure or shear stress on the latter as physical stimuli. In this variant, the biological material 11 are disposed on a separate holder 37a so that they are in optimal alignment in relation to the liquid or the gas.

The partial picture in FIG. 9F shows a further variant of the unit for generating physical stimuli, in which a mechanical drive 38 is provided and is configured in such a way that it exerts pressure from both sides on the flexible carrier 32, as indicated by the two arrows, whereby the flexible carrier 32 is deformed, i.e., a bending stress is induced and is transmitted as physical stimulus to the biological material 11.

In the partial picture in FIG. 9G, the unit for generating physical stimuli comprises a rotary drive 40 which is disposed outside the chamber 2 in this embodiment. An axle, which leads into the interior of the chamber 2, transmits a rotational motion, which is indicated by the arrow, to a container 41 in which the biological material 11 is present. The biological material 11 is thus set in rotational motion and subjected to centrifugal force.

In the partial picture in FIG. 9H, the unit for generating physical stimuli is achieved by a magnet 42 which is disposed outside the chamber 2 and exposes the biological material 11 to a magnetic field.

In the partial picture in FIG. 9I, the unit for generating physical stimuli is achieved by a voltage generator 43 which is disposed outside the chamber 2 and supplies electrical pulses to the biological material 11 via a line 44.

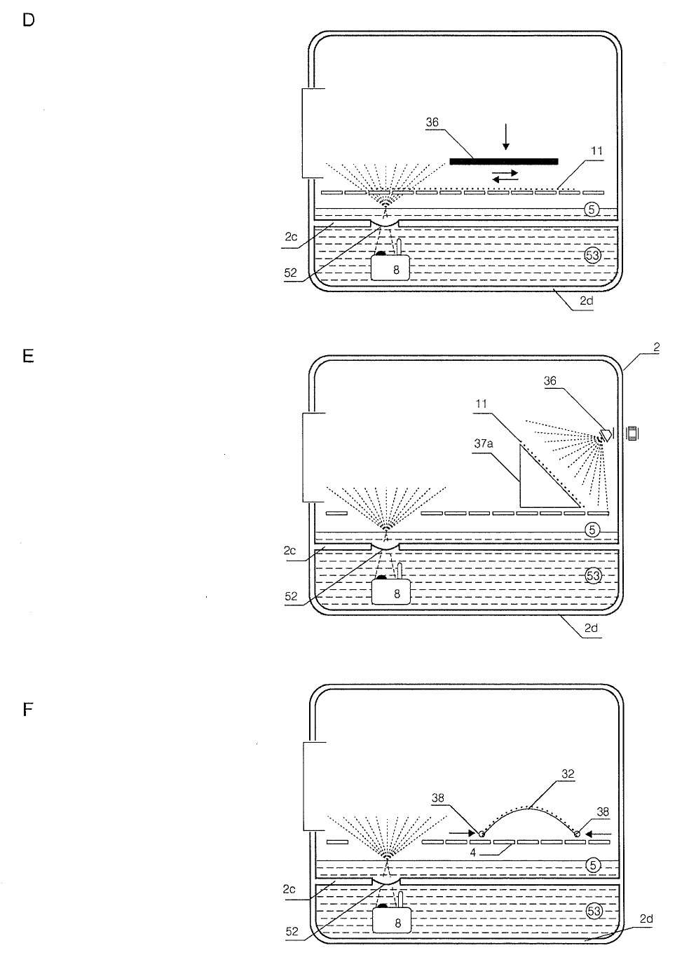

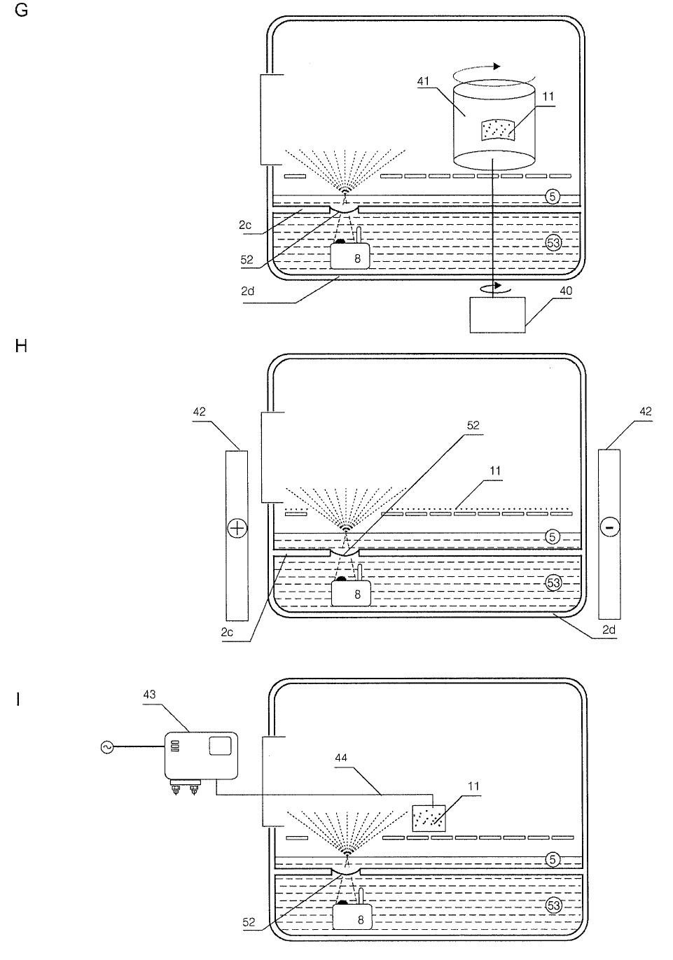

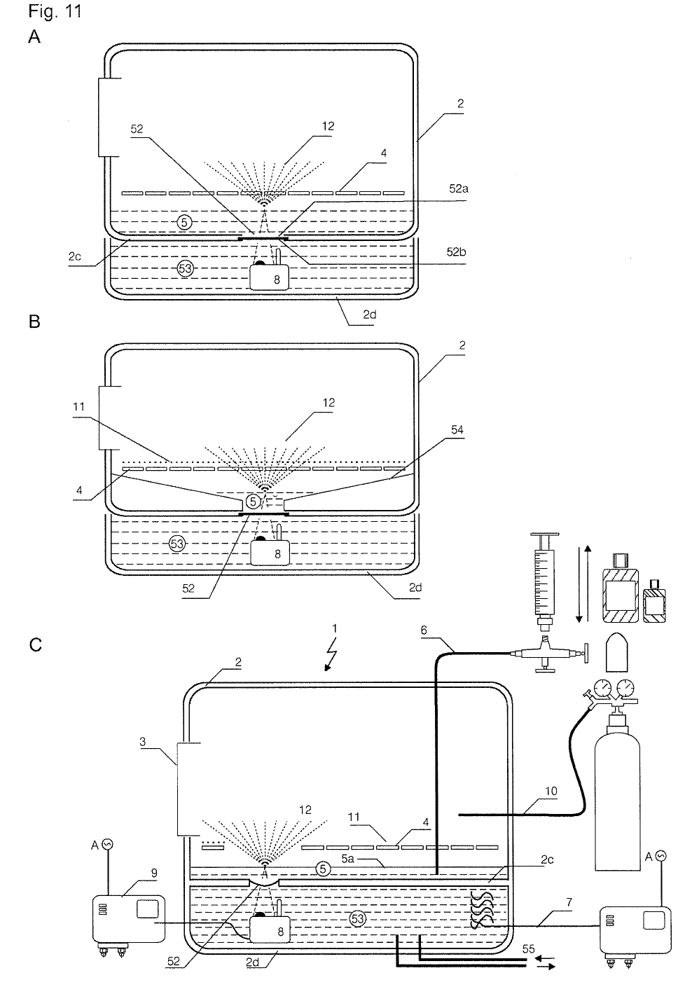

FIG. 11 shows diagrammatic representations of the device according to the invention in the form of a two-chamber system. The individual elements correspond to those of devices depicted in previous figures. The reference numbers have been retained where possible.

The chamber 2 depicted in the partial picture 11A comprises the culture medium 5. The chamber 2 comprises a base 2c into which is sealingly inserted the element 52 which transmits ultrasonic waves in the form of a plate consisting of an ultrasonic-transmitting material such as a metal. The culture medium 5 is in direct contact with a first surface 52a of the element 52. A medium 53 which transmits ultrasonic waves and is in direct contact with a second surface 52b of the element 52 is located in a second chamber 2d which is adjacent underneath. The second chamber 2d may also be referred to as transmission chamber. The ultrasonic generator 8 is located in the medium 53 which transmits ultrasonic waves and passes ultrasonic waves into the medium 53 which transmits ultrasonic waves. These are picked up from the second surface 52b of the element 52 which transmits ultrasonic waves and are passed onward via the first surface 52a of the element 52 which transmits ultrasonic waves into the culture medium 5 in the chamber 2. The aerosol or the mist 12 of culture medium 5 is produced in the chamber 2 thereby.

The partial picture in FIG. 11B depicts an embodiment of the device according to the invention in which a funnel-shaped reducer 54 is provided in the chamber 2. This reducer extends from the surface 52a of the element 52 which transmits ultrasonic waves in a funnel shape upwards in the direction of the biological material 11 present on the carrier 4. The base of the reducer 54 is in this case sealingly connected to the part of the base 2d of the chamber 2 which comprises the element 52 which transmits ultrasonic waves. The base of the reducer 54 can also be disposed directly on the element 52 which transmits ultrasonic waves. It is possible by this measure to reduce the amount of culture medium present in the chamber 2.

The partial picture in FIG. 11C depicts an overview of the device according to the invention corresponding to FIG. 1A, to which reference is made. The element 52 which transmits ultrasonic waves is depicted in the form of a diaphragm which is convex in the direction of the medium 53 which transmits ultrasonic waves. The temperature of the culture medium 5 is adjusted indirectly by the heating coil of the temperature control unit 7 which is introduced into the medium which transmits ultrasonic waves. The heat generated there is transmitted into the culture medium 5 via the base 2c of the chamber 2. Contamination of the culture medium 5 by the temperature control unit 7 is avoided thereby. Delivery and removal lines are depicted under reference number 55, through which the medium 53 which transmits ultrasonic waves can be delivered to the second chamber 2d, and removed therefrom, respectively.

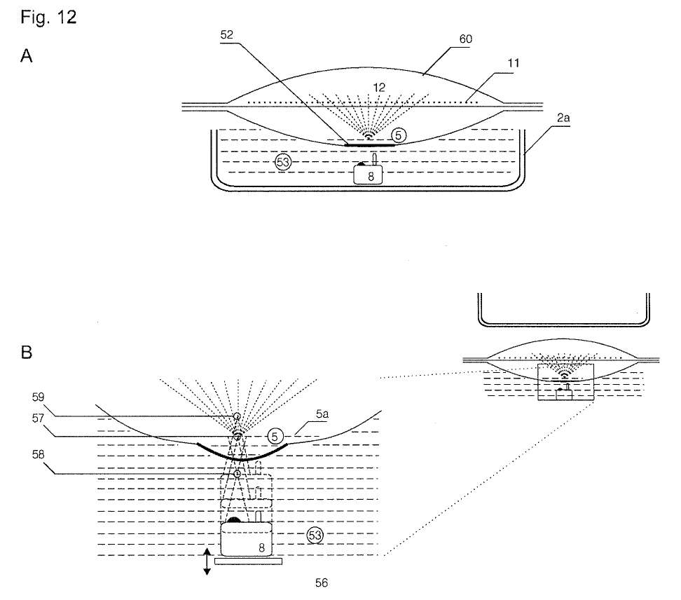

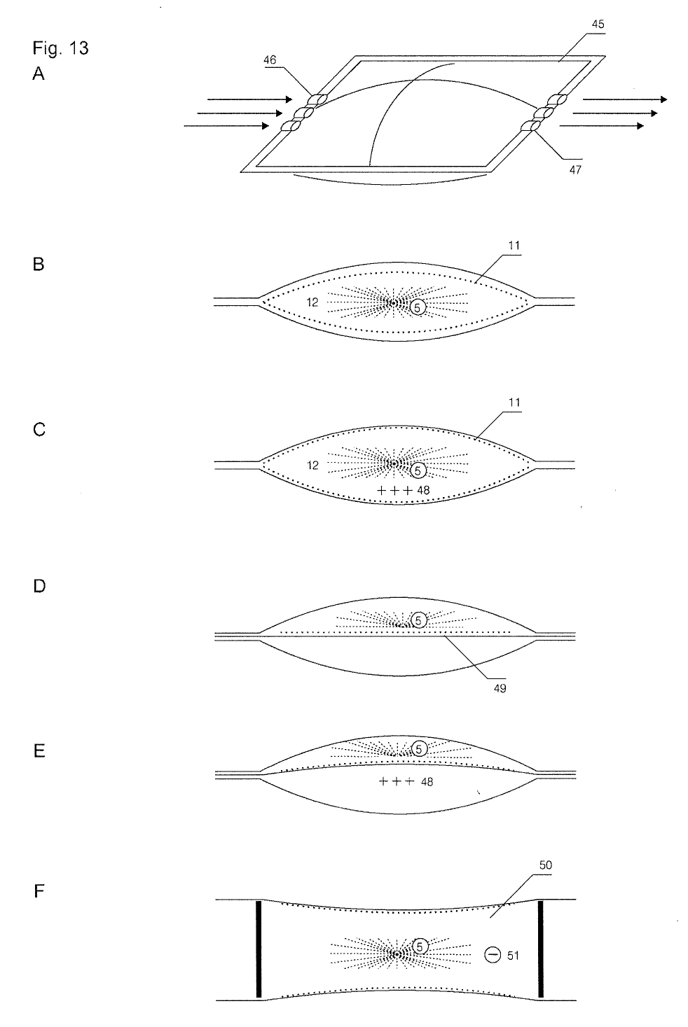

FIG. 12A depicts an embodiment of the device according to the invention in which the chamber 2 is achieved by a cushion 60. The cushion comprises in its base the element 52 which transmits ultrasonic waves in the form of a plate which is in direct contact via its first surface 52a with the culture medium 5 and by its second surface 52b with the medium 53 which transmits ultrasonic waves. The cushion 60 is described in detail in connection with FIG. 13 (provided with reference number 45 therein).

The partial picture in FIG. 12B depicts an enlarged detail of the region in which the ultrasonic is transmitted, starting from the ultrasonic generator 8 which introduces the ultrasonic waves into the medium 53 which transmits ultrasonic waves. They are passed onward into the culture medium 5 via the element 52 which transmits ultrasonic waves and is depicted in the form of a curved diaphragm. The ultrasonic points are depicted by reference numbers 57, 58, 59. The ultrasonic focus 57 is located on the surface 5a of the culture medium 5, leading to maximum nebulization of the culture medium 5 and droplet formation. The ultrasonic generator can have its position changed continuously, preferably in the vertical direction, by a platform 56, as indicated by the double arrow. When the ultrasonic generator 8 is lowered into the medium 53 which transmits ultrasonic waves, the ultrasonic focus 58 can be positioned in the medium 53 which transmits ultra-sonic waves. When the ultrasonic generator 8 is raised into medium 53 which transmits ultrasonic waves, the ultrasonic focus 59 can be positioned in the culture medium 5.

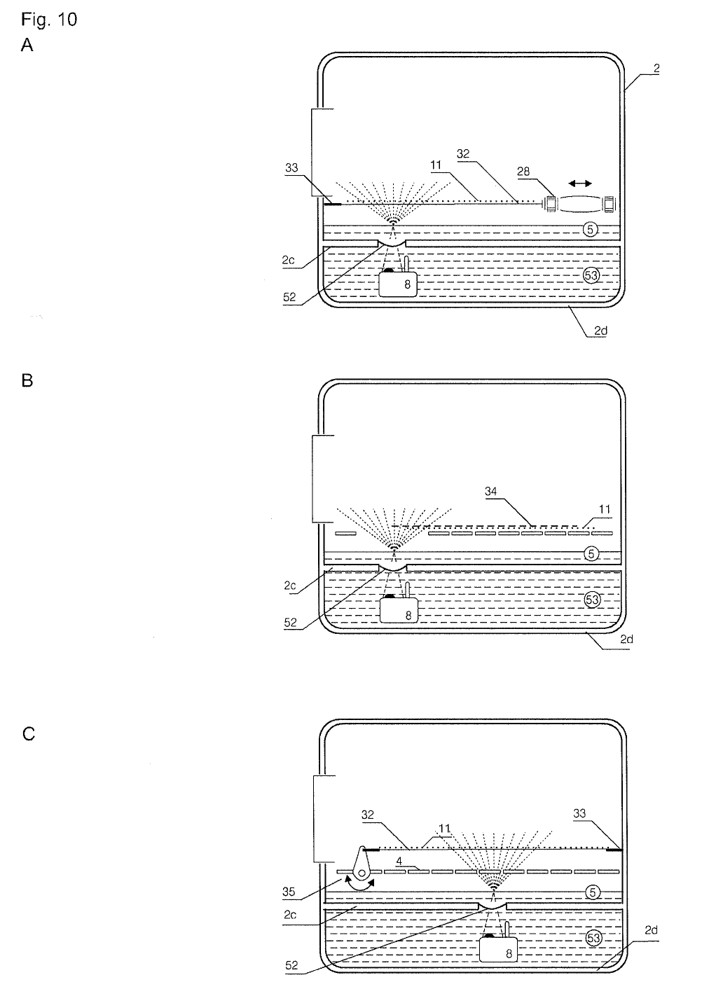

FIG. 10 depicts embodiments of the device according to the invention which include variants of the unit for generating physical stimuli. The individual elements correspond to those of the devices depicted in FIG. 9. The reference numbers are retained as far as possible. The base 2c of the chamber 2, the second chamber 2d, the element 52 which transmits ultrasonic waves and the medium 53 which transmits ultrasonic waves are depicted correspondingly. The partial picture in FIG. 10A shows the pneumatic drive 28 which may also be a hydraulic drive. The partial picture in FIG. 10B shows a variant of the physical stimulation of the biological material 11 where the contractile cells 34 are pro-vided and are in direct contact with the biological material 11 disposed on the carrier 4, and transmit the movement as physical stimulus to the material 11 through the contraction. The partial picture in FIG. 10C depicts a mechanical drive 35 as unit for generating physical stimuli. The partial picture in FIG. 10D depicts a further mechanical drive 36 which is in contact with the material 11 and exerts thereon mechanical pressure or mechanical shear stress as indicated by the arrows. The partial picture in FIG. 10E depicts a gas or liquid nozzle 37 as unit for generating physical stimuli. FIG. 10F shows a further variant of the unit for generating physical stimuli in which a further mechanical drive 38 is provided. In the partial picture in FIG. 10G, the unit for generating physical stimuli comprises a rotary drive 40 which is disposed outside the chamber 2 and the second chamber 2d in this embodiment. In the partial picture in FIG. 10H, the unit for generating physical stimuli is achieved by a magnet 42. In the partial picture in FIG. 10I, the unit for generating physical stimuli is achieved by a voltage generator 43.

FIG. 13 shows a pneumatic system in the form of a cushion 45 which is depicted diagrammatically in the partial picture in FIG. 13A. The culture medium 5 and any desired gas can be introduced through inlet apertures 46 into the interior of the cushion 45. The culture medium 5 or gas can be removed from the cushion again 45 through the outlet aperture 47.

As depicted in a partial picture in FIG. 13B, the interior of the cushion 45 or the inner wall of the latter can be colonized with the biological material 11. Incubation of the biological material 11 with the culture medium takes place through the mist 12 of culture medium 5 which is introduced into the interior of the cushion 45. The partial picture in FIG. 13B depicts the cushion 45 in the unstressed state so that no physical stimulus is exerted on the biological material 11.

In the partial picture in FIG. 13C, the amount of culture medium 5 delivered in the form of the mist 12 or of gas through the inlet aperture is increased and/or the amount removed through the outlet aperture 47 is reduced, so that a superatmospheric pressure 48 (+++) is produced in the interior of the cushion 45. The biological material 11 is thereby subjected, comparable to the variant depicted in FIGS. 9E and 10E, to a pressure or shear stress and simultaneously through the deformation of the wall of the cushion 45 to a bending stress corresponding to the variant depicted in FIGS. 9F and 10F.

The partial pictures in FIG. 13D and FIG. 13E correspond to the partial pictures 9B/10B and 9C/10C, but in this case the cushion 45 is designed as two-chamber cushion by providing a further flexible membrane 49 inside. It will be appreciated that any number of chambers can be provided by providing any number of additional, flexible membranes inside the cushion 45.

The partial picture in FIG. 13F depicts an alternative design of the cushion 50 where a subatmospheric pressure 51 (−) prevails inside through evacuation of gas and is transmitted as physical stimulus to the biological material 11.

### Example 2

The inventors have cultivated and generated various biological materials with the method according to the invention. In parallel, the same biological material was treated by conventional cultivation methods.

Cells of the sarcoma cell line SaOs2 were cultivated for five days according to the invention in a culture medium mist generated by ultrasonic and, in parallel, in a conventional way by covering with a layer of culture medium, i.e. submerged. It emerged from this that the cell density was similar in both mixtures.

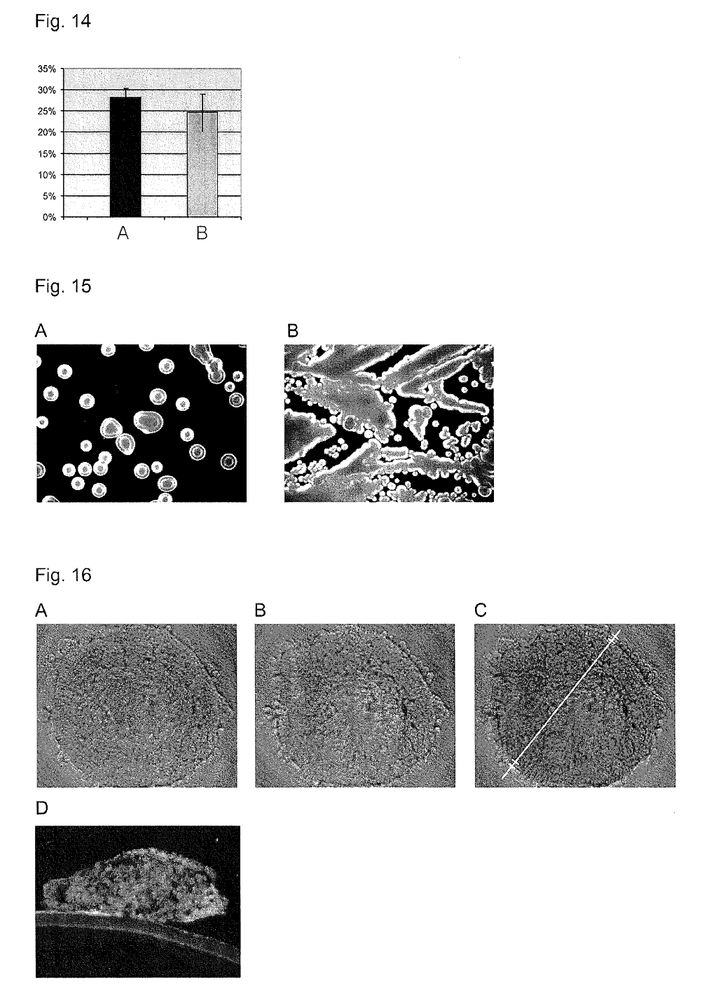

To determine the mitotic activity, the cells were stained with bromode-oxyuridine (BrdU), which becomes incorporated into the DNA of proliferating cells. In both mixtures, the stained cells were counted and the mitosis rate was determined. The result is shown in FIG. 14. The result shown on the left is for the cells exposed to vapor according to the invention (A) and that on the right is for the cells treated under submerged conditions (B). It emerged from this that the cells exposed to vapor according to the invention exhibit a greater proliferation activity (about 28% mitosis activity) than the cells which underwent conventional submerged cultivation (about 24%).

The treatment according to the invention therefore has positive effects on the vitality of biological cells by comparison with conventional cultivation methods.

In a subsequent experiment, a bacterially contaminated culture medium was used and was introduced as medium vapor into the device according to the invention for 6 h. Samples were subsequently taken of the deposited vapor. In parallel, the contaminated medium was incubated as liquid culture, i.e., not nebulized, under conditions which were otherwise the same, likewise for 6 h. Samples were also taken from the last mixture. The samples from the two mixtures were cultivated on an agar nutrient medium for 24 h. The bacterial growth in both mixtures was determined and compared with one another. As is evident in FIG. 15, the culture from the deposited vapor showed a significantly lower bacterial count (A) than the culture from the conventional liquid culture (B). This shows that bacteria were to a large extent killed in the method according to the invention.

The risk of bacterial contamination of the culture medium is thus distinctly reduced with the method according to the invention and the device according to the invention compared with conventional cultivation methods.

A further experiment investigated whether intestinal tissue treated according to the invention retains its physiological functionality. The parameter used for this purpose was the ability of the embryonic intestinal tissue to undergo spontaneous contraction. Intestinal tissue removed from the mouse embryo in developmental stage 14 (El4) was treated on a Millipore carrier membrane according to the invention with delivery of growth factors for five days and then the contractility was investigated. The result is depicted in FIG. 16. It was possible to show that the three-dimensional intestinal tissue culture which formed after this culturing period exhibited spontaneous physiological muscle contractions: (A)=relaxed; (B)=contracted; (C)=superimposed. It was possible to demonstrate by the subsequent histological investigations with diaminephenylindole dihydrochloride (DAPI) and phalloidin, which label living cells by binding respectively to DNA and the cytoskeleton, that the cells were vital (D).

This experiment confirms that the method according to the invention has positive effects on the vitality of cultivated and generated three-dimensional biological tissues which are not to be observed with conventional cultivation methods.

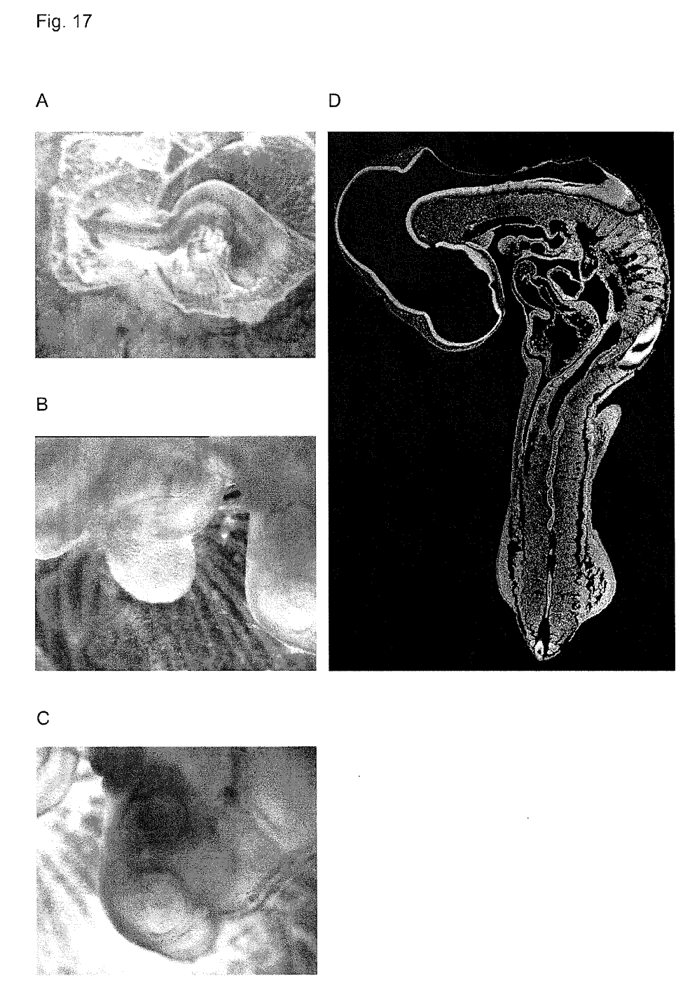

In a further experiment, explanted chick embryos in developmental stage 12 (E12) were cultivated on a membrane according to the invention in the medium mist with delivery of growth factors via the medium as physiological stimulus, and the development, i.e. the generation, was observed. The result is depicted in FIG. 17.

The embryo developed completely normally (A) within 48 h. At the end of the experiment in stage 18 of embryonic development it was possible to observe that a blood circulation with flowing erythrocytes had developed (C) and the heart contracted physiologically (B). Histological investigations confirmed the normal organ and tissue development of the embryos which had undergone ex ovum cultivation and generation (D). Control groups in the conventional incubator using conventional cultivation methods showed no vitality.

This experiment demonstrates that the inventors have succeeded for the first time in providing a method and a device with which it is possible by using modern methods of tissue engineering to cultivate and generate highly complex biological units such as embryos, which has not to date been possible with conventional cultivation methods.

What is claimed is:

1. A device for the cultivation and generation of biological material which comprises:
a unit for generating ultrasonic waves,
a chamber able to receive a biological material and a culture medium, comprising an element which transmits ultrasonic waves, comprising:
a first surface and
a second surface,
wherein the element is disposed in such a way that the first surface can make contact with the culture medium and the second surface can make contact with a medium which transmits ultrasonic waves and is disposed outside the chamber, and wherein the unit for generating ultrasonic waves is disposed outside the chamber in such a way that from these ultrasonic waves can be introduced into the medium which transmits ultrasonic waves, and be transmitted by the element into the culture medium.

2. The device of claim 1, wherein said unit for generating ultrasonic waves is designed so that it can form an ultrasonic focus which can be positioned in said culture medium.

3. The device of claim 2, wherein said unit for generating ultrasonic waves is designed so that it can alter the position of said ultrasonic focus in the culture medium in such a way that it coincides with said surface of said culture medium.

4. The device of claim 1, wherein said element which transmits ultrasonic waves is disposed in the base of said chamber and is selected from the group consisting of: a plate, a sheet or a diaphragm, consisting of a material which is selected from the group consisting of: ceramic, glass, minerals, metal, plastics and metal alloy.

5. The device of claim 1, wherein said plate, sheet or diaphragm comprises a thickness which is about 0.025 mm.

6. The device of claim 1, wherein said medium which transmits ultrasonic waves is a liquid or a gel.

7. The device of claim 1, wherein it further comprises a second chamber which is able to receive said medium which transmits ultrasonic waves and/or capable of at least partial reception of said unit for generating ultrasonic waves.

8. The device of claim 7, wherein said second chamber is disposed so that at least one boundary wall of said second chamber comprises said element which transmits ultrasonic waves.

9. The device of claim 1, wherein it further comprises a unit for generating physiological and/or physical stimuli acting on said biological material.

10. The device of claim 9, wherein said unit for generating physiological stimuli is realized by a device for delivering growth factors into said culture medium and/or by a biological or non-biological matrix which can make contact with said biological material.

11. The device of claim 9, wherein said unit for generating physical stimuli comprises a pneumatic, hydraulic or mechanical drive, or a rotary drive, which are able to set said biological material in motion.

12. The device of claim 9, wherein said unit for generating physical stimuli is realized by said chamber being a cushion whose shape can be altered by applying pressure.

13. The device of claim 9, wherein said unit for generating physical stimuli comprises means able to make contact with said biological material and exert mechanical pressure thereon.

14. The device of claim 9, wherein said unit for generating physical stimuli comprises a gas or liquid nozzle via which gas or liquid pressure can be exerted on said biological material.

15. The device of claim 9, wherein said unit for generating physical stimuli comprises a voltage generator able to deliver electrical pulses to said biological material.

16. The device of claim 9, wherein said unit for generating physical stimuli comprises a magnet able to expose said biological material to a magnetic field.

17. The device of claim 9, wherein said unit for generating physical stimuli comprises contractile biological cells able to make contact with said biological material.

18. A method for the cultivation and generation of biological material which comprises the following steps:
(a) provision of biological material and culture medium in a chamber which comprises an element, which transmits ultrasonic waves, and which comprises:
a first surface and
a second surface,
wherein said element is disposed in such a way that said first surface can make contact with said culture medium and said second surface can make contact with a medium which transmits ultrasonic waves and is disposed outside said chamber;
(b) introduction of ultrasonic waves into said medium which transmits ultra-sonic waves in such a way that they are transmitted by said element into said culture medium.

19. The method of claim 18, wherein said ultrasonic waves are introduced into said transmitting medium in step (b) in such a way that an ultrasonic focus results in said culture medium or in said medium which transmits ultrasonic waves.

20. The method of claim 19, wherein said ultrasonic waves are introduced into said culture medium in step (b) in such a way that said ultrasonic focus coincides with said surface of said culture medium.

21. The method of claim 18, wherein a physiological and/or physical stimulation of said biological material takes place in a further step (c).
